(19) United States
(12) Patent Application Publication
(10) Pub. No.: US 2009/0061507 A1
(43) Pub. Date: Mar. 5, 2009

(54) Fluorescence-based lateral flow device with improved sensitivity

(76) Inventor: Winston Z. Ho, Hacienda Heights, CA (US)

Correspondence Address: Winston Z. Ho, 14541 Langhill Drive, Hacienda Heights, CA, 91745, US

(21) Appl. No.: 11/974,358

(22) Filed: Oct. 12, 2007

Related U.S. Application Data
(60) Provisional application No. 60/853,896, filed on Oct. 23, 2006.

(51) Int. Cl. C12M 1/34; G01N 30/02

(52) U.S. Cl. 435/288.6; 422/70

(57) ABSTRACT

The invention describes how to use nanometer scale fluorescence particles as a label material for fluorescence lateral flow device application. The utilization of the nanoparticles instantly increases the fluorescence intensity by thousands to millions of times. The resulting signal enhancement not only significantly increase sensitivity for analyte detection, but also makes it possible to use low power light sources for illumination and low cost detectors for fluorescence detection.

### RELATED APPLICATION

The present application claims the benefits of Provisional application Ser. No. 60/853,896 filed Oct. 23, 2006, entitled “Fluorescence-based membrane strip and system”.

### FIELD OF THE INVENTION

The invention is related to lateral flow devices that utilize optical detection mechanism to quantify analyte with improved sensitivity and high precision for immunoassay and nucleic acid chemistry. Device and methods thereof are disclosed for easily and rapidly analyzing samples utilizing fluorescence detection methods in membrane chromatography. More specifically, the device uses fluorescence nanoparticles as a label agent to increase the emission intensity for low quantity of analyte detection.

### BACKGROUND OF THE INVENTION

Home test lateral flow strips or chromatographic strips are the most commonly used pregnancy tests. Purchased over the counter, these tests are so simple and can be performed by a woman in privacy of her home. Most strips provide a single test and use a color or absorption-based immunochromatographic strategy. Immunochromatography color strips are developed for rapid testing, but are based on visual inspection or absorption measurement, and have been reported to have poor sensitivity and accuracy. Fluorescence-based lateral flow strip, on the contrary, offers much better sensitivity. In the fluorescence-based measurement, the detection limit is based on the number of fluorescence photon been generated, instead of the change of color, from gold (Au) particles, in the conventional absorption-based strip. It is known that fluorescence measurement improves sensitivity by one to two orders of magnitudes in comparison with color-based measurement. The fluorescence-based chromatographic strip consists of a nitrocellulose lateral flow membrane integrated with an application pad, a conjugate release pad, and an absorbent pad. The application pad made of Whatman PlasmaSep membrane (Fairfield, N.J.) is placed over the lateral flow membrane and separates sample residue, such as cells, from the extracted fluid sample. The extracted sample and other compounds are transferred across the lateral flow membrane by chromatographic mechanism.

Different fluorophores have been developed as a label or tag for biological, biochemical, and clinical purposes. The intensity of the fluorescence is used for quantifying the analyte concentration. The criteria of the fluorescence label are:
1. It should be able to be excited with low cost light source;
2. The emission stoke shift should be as large as possible in order to filter out the excitation light;
3. The quantum yield of the fluorescence molecule must be large; and
4. The fluorescence intensity per conjugate molecules, such as antibody conjugate, must be amplified as much as possible.

Although fluorescence is more sensitive than absorption, conventional fluorescence is still not sufficient for many applications, especially for low trace of analyte detection. The reason is that each analyte is conjugated to one antibody, and each conjugate antibody is labeled with only one fluorophore. So the relationship is one analyte to one fluorescence molecule. It is known that it is difficult to detect a few molecules, due to the sensitivity of optical detectors. The rule of the thumb is that at least 1 million fluorescence molecules are needed in order to generate sufficient fluorescence signal to be detected by a low cost optical detector. Therefore, in order to detect trace of analyte (<1 M molecules), significantly increase the fluorescence intensity is needed. One analyte must be conjugated with many fluorophores, as mentioned in the No. 4 criterion above. This invention is to make the labeled fluorophores in the form of nanometer scale particles or spherical beads. Each nanoparticle or nanobead, with a dimension of 1 nanometer to 1,000 nanometers, may consist of thousands or millions of fluorophores that will generate fluorescence signal several orders of magnitude larger in compared to a single fluorophore.

The fluorescence molecules are impregnated in the polymer or latex medium at “maximum load” concentrations. The advantage of impregnating fluorophores into the nanobead is that it instantly amplifies fluorescence signal by thousand or million times. When a nanoparticle is excited by an external light source, it generates brighter fluorescence than that from a single molecule. This has significant implication in terms of commercial products. First, it is become possible to use low power and inexpensive light source for illumination. High power laser is no longer needed. Second, low cost detectors can be used. Expensive and bulky photon multiplier tube is not needed. These factors are critical for compact, handheld, and inexpensive devices, which can open up many applications, such as point of care testing (POCT). The particle matrix also provides better protection from photolytic degradation and maintains a longer shelf life. The latex beads, when treated with plasma or chemicals can generate functional groups, such as carboxyl or hydroxyl groups, on the surface. Thus beads can be covalently coupled to proteins or nucleic acids with conventional chemistry.

The technology can be used in point of care testing for rapid testing. Not all-clinical testing requires rapid test results or a compact portable system; however the majority of applications certainly do demand quick results and easy accessible. From the application point of view, one can divide the market into three product groups:
1. Urgent Diagnosis: Emergency room applications such as patients complaining of chest pains (troponin I, CK-MB, and myoglobin,) or exhibiting signs of a drug overdose (amphetamine, cocaine, opiates, etc,) require an almost instantaneous diagnosis.
2. Critical Detection: Patients with Hepatitis B, Hepatitis C, Chlamydia, or Gonorrhea in an emergency room or doctor's office require an accurate and immediate diagnosis to ensure long-term health.
3. Intense and Recurring Monitoring: Applications in surgery rooms (PTH monitoring, for example) or fertility treatment offices (LH and estradiol level monitoring) patients receiving anticoagulation drugs, and the use of therapeutic drugs (theophylline, methotrexate, etc,) require testing that is always accurate.

The broad base of customers and demands include: physician offices, bedside and near-patient testing, emergency rooms & intensive care units, blood banks, rural hospitals & nursing facilities, military & field application, and small & mid-size hospitals.

### SUMMARY OF THE INVENTION

The present invention is directed to a lateral flow device that utilizes fluorescence detection mechanism to quantify analyte with improved sensitivity and high precision. Device thereof are disclosed for easily and rapidly analyzing samples based on fluorescence immunoassay and nucleic acid chemistry in membrane chromatography. More specifically, the apparatus utilizes fluorescence nanoparticles as a label agent to amplify fluorescence intensity for low quantity of analyte.

One object of the present invention is to provide signal amplification with fluorescence nanoparticle to instantly increase the fluorescence intensity by several orders of magnitude.

One object of the present invention is to provide lateral flow devices with low cost and compact light source and detector as a detection system, and still be able to detect low trace of analyte.

Some aspects of the invention provide a lateral flow device for performing biological assay comprising a chromatographic strip consisting of a support membrane pad, wherein a sample application pad, a fluorescent-labeled conjugate release pad, and an absorbent pad are positioned in series on a top surface of the support pad; fluorescence nanoparticles embedded within the conjugate pad; and an analyte entering site adjacent the sample application pad, wherein at least one analyte is used for performing the biological assay. In one embodiment, the analyte is a pathogen, antigen, or a virus.

In one preferred embodiment, the lateral flow device further comprises a compact light source and a detector as a detection system for an analyte entering the site. In another preferred embodiment, the lateral flow device further comprises a microprocessor that controls the sequence of light illumination and measures the resulting fluorescence. In still another embodiment, the lateral flow device comprises a micro scanner mechanism that is installed as an auxiliary unit to the device, the scanner mechanism being for multiple sensors identification.

In one embodiment, the nanoparticles used in the present lateral flow device for performing biological assay are in the range of 100 nm to 900 nm, preferably in the range of 200 nm to 800 nm, and most preferably in the range of 200 nm to 400 nm. If the fluorescence particle is too big, it may be too heavy or too bulky, and are not able to migrate through the lateral flow membrane.

In one embodiment, the nanoparticles used in the present lateral flow device are excitable with a low cost diode laser at 630-650 nm, the nanoparticles generating strong fluorescent signal that is more than 50 nm red shift from the excitation laser light source. In an alternate embodiment, the nanoparticles are excitable with a red diode laser at 635 nm and 10 mW.

The biological assay by using the present lateral flow device includes an assay for detecting microorganisms of Streptococcus pyogenes, for determining concentration of adenovirus, or for determining presence of influenza A.

Some aspects of the invention provide an array disk comprising a plurality of the lateral flow devices disclosed herein, wherein each the lateral flow device is configured and arranged from a center of the disk radially outwardly toward a peripheral of the disk.

Prior to this invention, POCT devices are commonly lack of sensitivity. No system demonstrates the same performance: sensitivity, reliability, and precision based on a compact or handheld system. One of the objects of the present invention is to embed fluorescence nanoparticles in the lateral flow strips; therefore, a POCT device can achieve same performance as the large workstation or high-end laboratory instrumentation.

The present invention has the advantage of offering high sensitivity for lateral flow strips, and providing accurate and reproducible results. It should be understood, however, that the detail description and specific examples, while indicating as preferred embodiments of the present invention, are given by way of illustration and not of limitation. Further, as it will become apparent to those skilled in the art, the teaching of the present invention can be applied to devices for measuring the concentration of a variety of body fluidic samples in various platforms or configurations.

### BRIEF DESCRIPTION OF THE DRAWINGS

For a full understanding of the scope and nature of the invention, as well as the preferred mode of use, reference should be made to the following detailed description and read in conjunction with the accompanying drawings. In the following drawings, like reference numerals designate like or similar parts throughout the drawings.

FIG. 1 show (a) Lateral flow fluorescence sandwiched immunoassay for sample application (top); (b) Sample migration and analyte (Ag) binding with the antibody label bead to form (Ag-Ab*) (middle); and (c) Fluorescent signals from (anti-control) control at the control zone and analyte conjugates (Ab-Ag-Ab*) at the target line (bottom).

FIG. 2 shows some of the fluorescence-based nanoparticle Streptococcus A testing strips that were fabricated.

FIG. 3 illustrates the fluorescence nanoparticle that is excited by red light sources (630-650 nm) (left coordinate) and generate fluorescence (right coordinate) at near IR (720 nm).

FIG. 4 shows the data of fluorescent signal versus various dilutions of FITC fluorescence latex beads from the stock solution: top curve 0.1% (5×10⁴ beads), middle curve 0.01% (5×10³ beads), and bottom curve 0.001% (5×10² beads) on membrane strips.

FIG. 5 shows the data of nanoparticle fluorescence spectra for various concentrations (no. of microorganism) of streptococcus pyogenes tests.

FIG. 6 shows the system's sensitivity analysis: fluorescence intensity as a function of the adenovirus concentration.

FIG. 7 shows the system's sensitivity analysis: fluorescence intensity as a function of the influenza A concentration.

### DETAILED DESCRIPTION OF EMBODIMENTS OF THE INVENTION

The present description is of the best presently contemplated mode of carrying out the invention. This description is made for the purpose of illustrating the general principles of the invention and should not be taken in a limiting sense. The scope of the invention is best determined by reference to the appended claims.

Fluorescence Nanoparticles-Based Strip

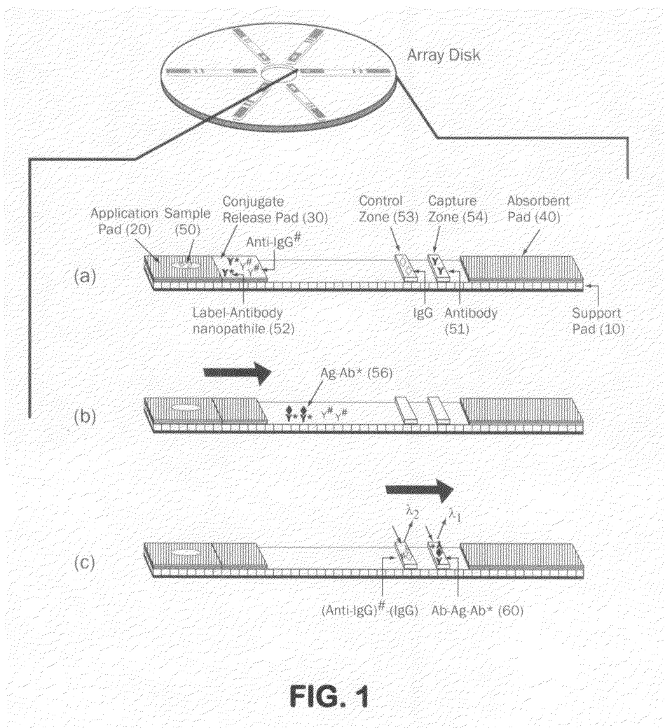

Lateral flow devices are designed in different configurations, such as microflow channels, microfluidics, electrophoresis, or membrane strip, which can use fluorescence nanoparticles as analytical devices. The most common lateral flow device is the membrane-based chromatographic strip. The lateral flow device 5 (FIG. 1) consists of four parts: the support membrane pad 10, the sample application pad 20, the fluorescent-labeled conjugate release pad 30, and the absorbent pad 40. To assay, the sample is added to one end of the strip with the sample application pad 20. The sample containing target analyte is filtered through the application pad. Through the capillary effect, the sample is moved along a flow path with fluorescence labeled conjugates.

In a sandwich immunoassay form at, for example, the sample analyte 50 or antigen (Ag) migrates through the application pad and into the supported membrane pad. The analyte then binds to the fluorescence labeled conjugate antibody (Ab*) nanoparticles 52 to form Ag-Ab* complex 56. The symbol “*” is fluorophore in the form of nanoparticles, which is then captured by the immobilized antibody 51, and forms (Ab-(Ag-Ab*)) complex 60 at the capture zone 54. At the same time the control fluorescent label, containing the known amount of anti-goat antibody, are trapped by the immobilized goat IgG at the control zone 53 and form (Ab*)-Ag, while unreacted material is transferred to an absorbent pad at the end of the strips. The absorbent pad at the distal end of the strip, aids in drawing the sample via the capillary effect through the strip. Fluorescent signals from the capture zone are measured and normalized against those from the control zone; the normalized signal is correlated to the analyte concentration. The internal control of the assay corrects for membrane variability, light source, and detector fluctuations using internal calibration.

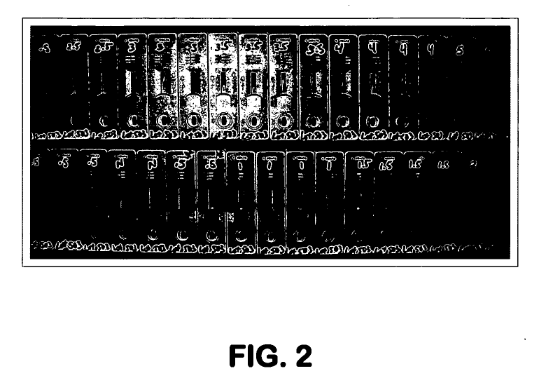

The selection of control antibodies depends on the animal in which the antibodies were generated. Ideally the control antibody has no cross reactivity with the antibodies bound to the capture zone. More antigens in the sample yield more antigen-antibody binding. The fluorescence signal or ratio (analyte to signal-control) increases while increasing analyte concentration. The dimension of the lateral flow membrane strip is about 3×25 mm. The lateral flow strip is assembled in a cassette which is about 20×70 mm. An array of strips can be configured and arranged from a center of said disk radially outwardly toward a peripheral of said disk. FIG. 1 show (a) Lateral flow fluorescence sandwiched immunoassay for sample application (top); (b) Sample migration and analyte (Ag) binding with the antibody label bead to form (Ag-Ab*) (middle); and (c) Fluorescent signals from (anti-control) control at the control zone and analyte conjugates (Ab-Ag-Ab*) at the target line (bottom). FIG. 2 shows some of the Streptococcus A testing strips that were fabricated and constructed in the cartridge for analytical characterization.

The pads used in the lateral flow membranes are used to transport the fluids, impregnate the labeled antibody, and binding the capture probes. It has a significant effect upon the protein binding observed in a rapid assay, with three key factors affecting performance: pore size, membrane post treatment, and membrane type. They are further discussed below.

Pore size generally has a direct relationship with the wicking rate and the protein-binding property of a membrane. It has major effect on the migration of nanoparticle. In the absence of any special treatment, membranes with larger pore sizes generally have a faster wicking rate but a lower surface area, which results in lower protein binding. Pore size measurement differs between membrane manufacturers, so this attribute should only be used to compare products within a single vendor's suite.

Membrane post treatment may include the addition of surfactants, or a post-washing step to remove the inevitable dust that is present on the membrane surface after manufacture. Treatments are unique to individual manufacturers and it should be established whether these have introduced any materials that may have an effect on protein binding, wicking or membrane aging, and capillary effect of nanoparticles.

Some membranes have been specifically developed for particular applications. Hydrophobic and hydrophilic properties of the membrane can significantly affect the protein binding and fluid kinetics.

Some aspects of the invention relate to a lateral flow device for performing biological assay comprising: (a) a chromatographic strip consisting of a support membrane pad, wherein a sample application pad, a fluorescent-labeled conjugate pad, and an absorbent pad are positioned in series on a top surface of the support pad; (b) fluorescence nanoparticles embedded within the conjugate pad; and (c) an analyte entering site adjacent the sample application pad. As illustrated in FIG. 1, one aspect of the invention relates to an array disk comprising a plurality of the lateral flow devices disclosed herein, wherein each the lateral flow device is configured and arranged from a center of the disk radially outwardly toward a peripheral of the disk.

Fluorescence Nanoparticles

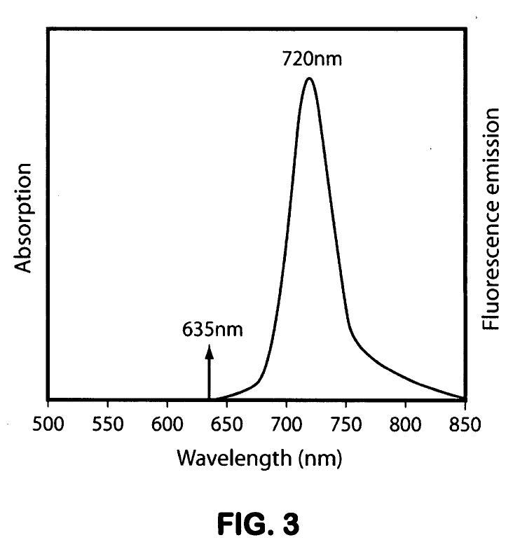

Several kinds of fluorescent particles are commercially available. TransFluroSpheres particles at sizes of 100 nm (Ex 633 nm/Em 720 nm) and 300 nm (Ex 630 nm/Em 720 nm) can be obtained from Invitrogen. Nanospheres particles at the size of 200 nm (Ex 350 nm/Em 613 nm) and 300 nm (Ex 660 nm/Em 700 nm) can be obtained from Duke Scientific. In a series characterization experiments, TransFluroSpheres particles (Ex 630 nm/Em 720 nm) at the size of 300 nm were chosen based on the sensitivity and the adaptability to the lateral flow membrane assay (FIG. 3). TransFluroSpheres can be not only excited with low cost diode laser at 630 nm, but also generate strong fluorescent signal, which is about 90 nm red shift from the excitation light source. The large red shift offers low background signal, thus gives large signal to background ratio. Magspheres offers a series of fluorescence particles, at sizes between 200 nm and 800 nm, with green, yellow, and orange fluorescence.

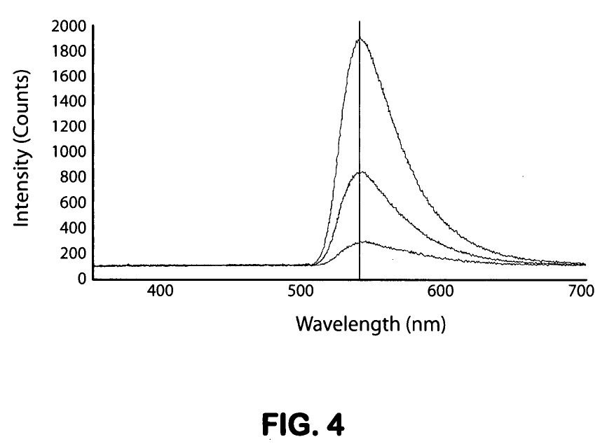

The FITC nanoparticles provide the primary amines of proteins to form the desired dye-protein conjugate. The absorption and fluorescence emission maximal of FITC-labeled protein is approximately 480 nm and 530 nm, respectively (FIG. 4). The carboxylated PS latex bead were diluted in a MOPS buffer (0.1M, Tween-20 0.1%, pH 4-6.0). The experiments were performed by dilution of the FITC bead stock solution (10M-50M beads/ml). The dilutions (0.1%, 0.01%, and 0.001%) were prepared to make up the concentrations for system tests. The FITC beads were directly applied onto the capture line of the strip. The typical spot sizes were 1-2 mm in width. The typical FITC bead concentration used in clinical testing is 0.1% with 10,000 to or 0.01% with 5,000 beads. Excellent signal-noise ratios were obtained on samples with a dilution of 0.001%, equivalent to a concentration of 100-500 beads/ml. Therefore, the system achieves a detection limit one to two orders of magnitude better than the typical clinical range. These fluorescence beads can be excited with inexpensive ultraviolet light source, such as mercury or xenon lamp.

Fluorescent Detection

The fluorescence nanoparticle-based device consists of optical components, light sources, optical filters, detectors, and scanning mechanism for highly sensitive detection. To ensure the system's compactness, robustness, and reliability, all system components was integrated with solid hardware. Newly developed laser diodes are inexpensive and have very stable output and a very long usable life. A red diode laser (635 nm, 10 mW) costs about $30, and was used to excite the fluorescence nano beads. The illumination was designed with direct illumination or through an optical fiber. The different light sources, such as LEDs, can also be coupled into the fiber and the illumination area. This opens the flexibility of using the device for different fluorophore excitation. The reflected fluorescence signals was collected through an optical filter, bifurcated optical fiber probe (fiber bundle) and delivered to the detector.

The microprocessor controls the sequence of light illumination and measures the resulting fluorescence. Only one detector is needed, which sequentially collects the fluorescence signal from the capture zone. Since the fluorescent wavelength is known, an optical filter, with a sharp cut-off and a high optical density (O.D.=4), is used to remove illumination light and pass through the fluorescent light. It is important to remove as much illumination light as possible. Illumination angle is critical to avoid the back reflection of excitation light. Low cost detector, such as CCD or photodiode, is sufficient for fluorescence detection. When the linear CCD and a grating are used, the system is able to detect the fluorescence spectroscopy. The collected signals obtained from the detector passed to PCs via A/D converters. The system and software are capable of performing real time normalization of background signals or control signals. The exposure time was set at 100 ms, while sample signals and control signals increased as a function of the integration time. The analog signal was fed into the analog-to-digital converter and then to the microprocessor for signal processing.

To detect the fluorescence from multiple sensing spots, the fluorescent reader can be facilitated with multiple light sources/detectors, or using a scanning mechanism that can scan the entire strip. A translation stage was built in with a miniaturized motor, which can be positioned at any place along the strip for optical excitation and detection. For current tests, two buttons (C for control and T for test) were setup for easy operation. The user can simply push any one of these two buttons and the scanner will automatically move the light source or cartridge to the correct positions for optical detection. Through a series of experiment, the system was optimized to achieve the most sensitive results with a large dynamic range.

The optical power of the diode laser was set at 5 mW at sensing spot and the signal integration time was set as 100 milliseconds. The laser spot size is 2 mm in diameter, and the angle between the optical probe and strip is designed to avoid the directed reflection of the excitation light source. The purpose of this configuration is to minimize the reflection from the excitation light. A micro scanner mechanism was installed in the system to support the cartridge. The scanner moves the cartridge, 2 inch in distance, for multiple sensors identification. Because the length of the membrane strip is 3 inch, it is possible to immobilize multiple probes on the same strip for simultaneous multiple pathogen diagnostics.

Streptococcus pyogenes Tests

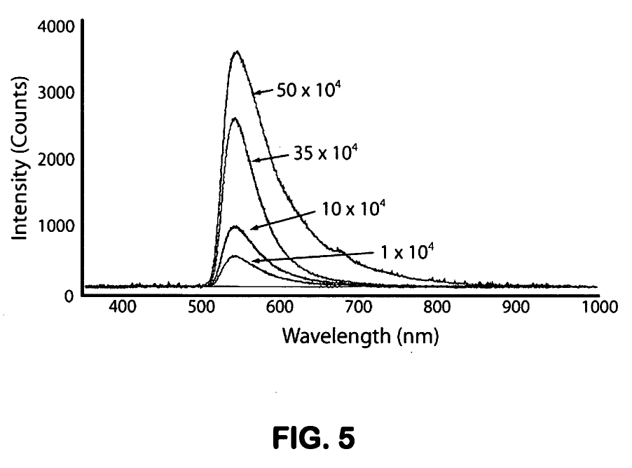

Monoclonal anti-streptococcus was conjugated to FITC-nanoparticles. This solution was applied to the conjugate release pad. Polyclonal anti-streptococcus A was immobilized at the capture line of the membrane in a concentration of 10 μg/line. The streptococcus samples were prepared with dilutions on the standard control sample from Quidel Corp. Samples (or sample titers) (150 μl) were loaded onto the sample pad. Streptococcus A, bound to the FITC-antibody nanoparticle on the release pad, migrated along the membrane. The complexes were captured at the capture line. The fluorescence intensity at the capture line was measured in order to determine the concentration of streptococcus. FIG. 5 shows the plot of fluorescence spectra for various analyte concentrations. The QuickVue test yielded positive results with specimens containing 50×10⁴ microorganisms per test. The microbiological agent is negative for gp. B, C, D, E, F, and G. The system's analytical sensitivity was based on the slope of the analyte titers (0.5×10³, 1.0×10³, 2.5×10³, 3.5×10³, 5.0×10³ Streptococcus pyogenes)—fluorescence response curve. Based on these tests, we have achieved a sensitivity of detecting 500 microorganisms of Streptococcus pyogenes.

Adenovirus Tests

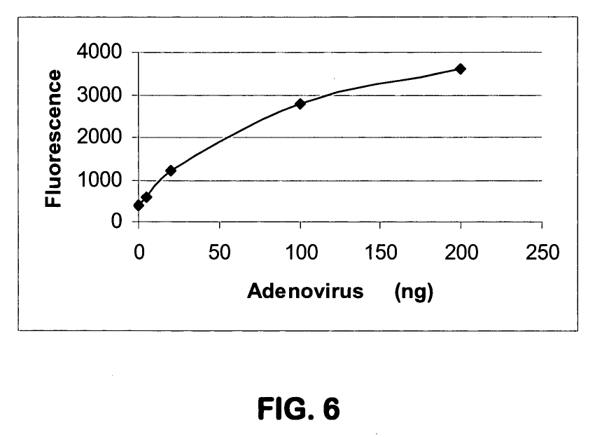

FIG. 6 shows the system's analytical sensitivity based on the slope of the analyte titers (0.1, 5.0, 20, 100, 200 ng Adenovirus). The samples were titered with a stock adenovirus (Fitzgerald 30-AA02, hexon from Adenovirus, Type 2) viral agent solution with 5.0 mg/ml protein concentration. It does not react with Parainfluenza 1, 2, or 3, influenza A & B or RSV. Monoclonal anti-adenovirus was conjugated to FITC-beads. This solution was applied to the conjugate release pad. The polyclonal anti-adenovirus was immobilized at the capture line of the membrane in a concentration of 10 μg/line. Samples (or sample titers) (150 μl) were loaded onto the sample pad. Adenovirus bound to the antibody on the release pad and migrated along the membrane. These complexes were captured at the capture line. The fluorescence intensity at the capture line was measured in order to determine the concentration of adenovirus. The total incubation time was 5 minutes.

Influenza A Tests

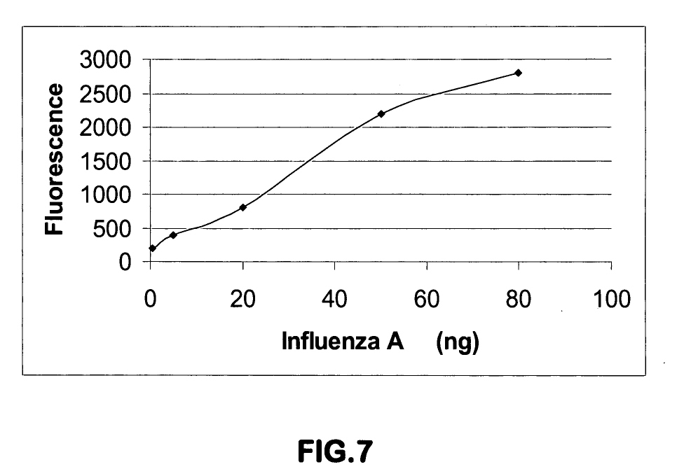

The viral agent samples were prepared with dilutions on the influenza A (Fitzgerald 30-A150) stock solution (4.9 mg/ml protein concentration). It does not react with Influenza B, RSV, adenovirus or Parainfluenza 1-3. Monoclonal anti-influenza A was conjugated to FITC-beads. This solution was applied to the conjugate release pad. Polyclonal anti-influenza A was immobilized at the capture line of the membrane in a concentration of 10 μg/line. Influenza A bound to the antibody on the release pad and migrated along the membrane. The PPS system's analytical sensitivity was based on the slope of the analyte titers (0.5, 5, 20, 50, and 80 ng)—fluorescence response curve, as shown in FIG. 7. Based on these tests, the system has achieved a detection limit of 0.5 ng influenza A.

The claim of the invention is:

1. A lateral flow device for performing biological assay comprising:
(a) a chromatographic strip consisting of a support membrane pad, wherein a sample application pad, a fluorescent-labeled conjugate release pad, and an absorbent pad are positioned in series on a top surface of said support pad;
(b) fluorescence nanoparticles embedded within said conjugate release pad; and
(c) an analyte entering site adjacent said sample application pad.

2. The device of claim 1, wherein said nanoparticles are in the range of 100 nm to 900 nm.

3. The device of claim 1, wherein said nanoparticles are in the range of 200 nm to 800 nm.

4. The device of claim 1, wherein said nanoparticles are in the range of 200 nm to 400 nm.

5. The device of claim 1 further comprising a compact light source and a detector as a detection system for an analyte entering said site.

6. The device of claim 5 further comprising a microprocessor that controls the sequence of light illumination and measures the resulting fluorescence.

7. The device of claim 5, wherein said nanoparticles are excitable with a low cost diode laser at 630 nm-650 nm.

8. The device of claim 5, wherein said nanoparticles are excitable with a low cost UV LED light source.

9. The device of claim 5, wherein said nanoparticles are excitable with a low cost diode laser, said nanoparticles generating strong fluorescent signal that is more than 50 nm red shift from said excitation laser light source.

10. The device of claim 5 further comprising at least one optical filter to remove the excitation light from said light source and transmit the resulting fluorescence from said nanoparticles.

11. The device of claim 5 further comprising a micro scanner mechanism that is installed as an auxiliary unit to said device, said scanner mechanism being for multiple sensors identification.

12. The device of claim 5 further comprising internal control at the control zone on the said support pad, and the control assay corrects for membrane variability, light source and detector fluctuations using internal calibration.

13. The device of claim 1, wherein the biological assay is for detecting microorganisms.

14. The device of claim 1, wherein the biological assay is for determining concentration of virus.

15. The device of claim 1, wherein the biological assay is for determining presence of influenza A.

16. The device of claim 1, wherein the biological assay is for determining presence of antigen.

17. The device of claim 1, wherein the biological assay is for determining presence of pathogen.

18. An array disk comprising a plurality of said lateral flow devices according to claim 1, wherein each said lateral flow device is configured and arranged from a center of said disk radially outwardly toward a peripheral of said disk.
